(19) United States
(12) Patent Application Publication
(10) Pub. No.: US 2013/0171224 A1
(43) Pub. Date: Jul. 4, 2013

(54) TREATMENT OF BIOFILMS

(71) Applicants: Steven Lane Percival, Chester (GB); Brian John Hamerslag, Cheshire (GB)

(72) Inventors: Steven Lane Percival, Chester (GB); Brian John Hamerslag, Cheshire (GB)

(21) Appl. No.: 13/574,351

(22) PCT Filed: Jan. 14, 2011
(86) PCT No.: PCT/GB11/00042

§ 371 Date: Mar. 20, 2013

(30) Foreign Application Priority Data
Jan. 21, 2010 (GB) 1000916.5

(51) Int. Cl. A01N 59/26; A61K 33/38; A61K 33/42; A01N 59/16

(52) U.S. Cl. CPC A01N 59/26; A01N 59/16; A61K 33/38; A61K 33/42
USPC 424/404; 424/604; 424/445; 424/409

(57) ABSTRACT

The combination of a first species which is a polyanionic compound and a second species which is an antimicrobial agent is used for the treatment of microbial biofilms and for the topical treatment of wounds. The combination may be incorporated in a wound dressing. The polyanionic compound may be a polyphosphate, such as an alkali metal polyphosphate, e.g. sodium hexametaphosphate. Examples of antimicrobial agents include metallic silver, silver compounds, iodine, PHMB (polyhexamethylene biguanide), acetic acid, chlorhexidine, aminoglycosides (e.g. amikacin, gentamicin, streptomycin and tobramycin), ansamycins, carbacephem, cephalosporins, glycopeptides (e.g. vancomycin), macrolides (eg clarithromycin), monobactams and sulfonamides).

The present invention relates to compositions, dressings and methods for the treatment of biofilms, particularly to inhibit, disrupt, kill and/or remove a microbial biofilm. The invention has particular (but not exclusive) application for the treatment of wounds.

Biofilms have been documented by the Centers for Disease Control (CDC) and National Institutes of Health (NIH) to account for 65% of all nosocomial infections and 80% of all known infections. Microbial biofilms develop when microorganisms attach to a surface and become encased within a three dimensional matrix of extracellular polymeric substances (EPS). They are medically and industrially important because they can accumulate on a wide variety of surfaces and become highly resistant to antimicrobial agents, the immune response and detergents and therefore pose a concern to public health.

Biofilms in the medical environment, particularly when found in or on indwelling medical devices are composed of Gram-positive or Gram-negative bacteria or yeasts. The specific bacteria that have commonly been isolated from medical devices, which have resulted in infections, have included the Gram-positive Enterococcus faecalis (E. faecalis), Staphylococcus epidermidis (S. epidermidis), Staphylococcus aureus (S. aureus), Streptococcus viridans (St. viridans) and the Gram-negative Escherichia coli (E. coli) and Klebsiella sp.

Chronic infections which are difficult, or impossible, to eliminate with conventional antibiotic therapies are known to involve biofilms. A partial list of the infections that have been shown to involve biofilms have included otitis media, prostatitis, cystic fibrosis pneumonia, necrotising fasciitis, osteomyelitis, peridontitis, biliary tract infection, struvite kidney stone and nosocomial infections¹

Tissue samples which have been taken from patients with dental caries, periodontitis and prostatitis have been shown to contain biofilm ‘markers’ such as bacterial microcolonies and EPS.² Biofilms harbouring multispecies of bacteria³ have also been documented in chronic wounds and implicated as the cause of an underlying sub-optimal infection and either delayed wound healing or non-healing of wounds. It has been estimated that approximately 2% of the population in the United States alone are experiencing a non-healing wound.⁴ The cost of this to the health service system and the patients quality of life are severe.

Consequently, as biofilms are responsible for recalcitrance in chronic wounds it is necessary to develop anti-biofilm compositions which are effective at killing microorganisms residing within a wound biofilm. Also it is important that anti-biofilm agents can disrupt and remove the EPS found within the biofilm. Such anti-biofilm compositions should be environmentally friendly, medically acceptable, effective at low concentrations and relatively economical to manufacture on a commercial scale.

According to a first aspect of the present invention there is provided the combination of a first species which is a polyanionic compound and a second species which is an antimicrobial agent for the treatment of microbial biofilms.

According to a second aspect of the present invention there is provided the combination of a first species which is a polyanionic compound and a second species which is an antimicrobial agent for the topical treatment of wounds.

According to a third aspect of the present invention there is provided a topically administrable wound treatment composition comprising a first species which is a polyanionic compound and a second species which is an antimicrobial agent.

According to the invention, we have established that the combination of a polyanionic compound and an antimicrobial agent is effective for the treatment of microbial biofilms. By such treatment we include killing or removing a microbial biofilm, inhibiting microbial biofilm formation, and disrupting an existing microbial biofilm. The combination is particularly effective for treatment of microbial biofilms in or on a wound. The combination may be in the form of a topically administrable wound treatment composition which comprises the polyanionic compound and the antimicrobial agent. Although the principal envisaged application of the present invention is in the field of wound treatment, other applications are possible. Thus, for example, the combination of the invention has application for the treatment of microbial biofilms on surfaces, e.g. household work surfaces.

The polyanionic compound may be in the form of a salt, e.g. an alkali metal salt.

In preferred embodiments of the invention, the polyanionic compound is a polyphosphate and the composition contains 0.1 to 200 mg/ml of the polyphosphate. The polyphosphate is preferably a sodium polyphosphate and most preferably sodium hexametaphosphate.

Polyphosphates (e.g. sodium hexametaphosphate) are anionic compounds which are able to chelate cations such as magnesium, calcium and manganese ions.⁵ In addition to this they are also considered to be weak antimicrobials and potent microbial sensitizing agents.⁶ Because of these characteristics of polyphosphate we have recognised that polyphosphates (such as sodium hexametaphosphate), when used in conjunction with antimicrobial agent, are powerful anti-biofilm agents. The concept is that the polyphosphate chelates metal ions and by removing, iron, calcium and magnesium from the biofilm will cause biofilm breakdown. Once the biofilm is broken down it no longer provides protection to the microbes and they will become more susceptible to the antimicrobial agents. In addition the polyphosphates are permeating agents which will enhance the uptake of antimicrobials by the microbes and will therefore enhance the efficacy of the antimicrobials.

Although polyphosphates are the polyanionic compounds of preferred choice for use in the invention, and other polyanionic compounds may be used and examples include polycarboxylic acids such as polyacrylic acid and polymethacrylic acid as well as polysulphonic acids (for example pentosan polysulfate, which is currently used to treat interstitial cystitis).

The antimicrobial agent may be selected from metallic silver, silver compounds, iodine, PHMB (polyhexamethylene biguanide), acetic acid, chlorhexidine and groups of antibiotics (Aminoglycosides (Amikacin, Gentamicin, Streptomycin, Tobramycin), Ansamycins, Carbacephem, Cephalosporins, Glycopeptides (Vancomycin), Macrolides (eg Clarithromycin), Monobactams, Sulfonamides). Silver compounds that may be used for the purposes of the present invention include (but are not limited to): silver sulphate, silver carbonate, silver nitrate, silver chloride, silver oxide, silver citrate, silver hydrogen citrate, silver dihydrogen citrate and silver salts of EDTA (ethylenediaminetetraacetic acid). Silver complexes may also be used, e.g. silver sodium hydrogen zirconium phosphate (available as AlphaSan). If metallic silver is used then nano-crystalline silver may be employed. A further possibility is for metallic silver to be provided as a coating on fibres and/or fabrics. Such silver coated fibres and/or fabrics have particular application for wound dressings (see also below).

The combination of the polyanionic compound and the antimicrobial agent may be applied to prevent or inhibit formation of a biofilm or to disrupt, kill and/or remove an existing biofilm in a wound. The combination has application for the treatment (including prophylactic treatment) of wounds under a wide range of circumstances. For example, the combination may be applied to a surgical incision, other forms of acute wound (e.g. resulting from an accident) or to a chronic wound. None limiting examples of wounds that may be treated by the combination of the invention include surgical wounds, burns, venous leg ulcers, arterial ulcers, diabetic ulcers, pressure ulcers, donor sites, traumatic wounds and cavity wounds.

In one embodiment, the invention provides a composition (containing the polyanionic compound and the antimicrobial agent) which may for example be formulated as a liquid, powder, lotion, gel, oil, ointment, gel, semi-solid formulation and aerosol spray. Such formulations may be produced in a conventional manner using appropriate carriers which are well known to a person skilled in the art.

The amount of the polyanionic compound (e.g. a polyphosphate) present in a composition in accordance with the invention may, for example, be in the range of 0.1-200 mg/ml, more preferably 0.1-100 mg/ml. For example, the amount of the polyanionic compound may be 40-60 mg/ml. The amount of the antimicrobial may be in the range 0.01 μg to 250 mg/ml, more preferably 1 μg to 250 mg/ml. If the antimicrobial agent is iodine then it may most preferably be used in an amount of 1 μg to 2500 μg/ml. Silver as the antimicrobial agent will typically be used in an amount of 1 mg-250 mg/ml. Antibiotic compounds as the antimicrobial agents will typically be used in a range of 1 μg to 1000 μg/ml.

The combination in accordance with the invention may be in the form of a wound dressing in which the antimicrobial agent and the polyanionic compound are provided separately or together within the wound dressing and/or on the wound contacting surface thereof.

Thus a further aspect of the invention provides a wound dressing which is intended to be applied to a wound to be treated and which comprises a substrate comprising the combination in accordance with the invention. Such a dressing is particularly convenient because it delivers the combination of the invention to the wound to be treated and simultaneously provides a dressing therefor. The wound dressing may, for example, be fibrous, a foam, a hydrocolloid, a collagen, a film, a sheet hydrogel or a combination thereof.

The wound dressing may be in the form of a layered dressing in which one or more layers of the dressing are formed at least in part or one or of; natural fibres, alginate, Chitosan, Chitosan derivatives, cellulose, carboxymethyl-cellulose, cotton, Rayon, Nylon, acrylic, polyester, polyurethane foam, hydrogels, hydrocolloids, polyvinyl alcohol, starch, a starch film, collagen, hylaronic acid and its derivatives, biodegradable materials, and combinations thereof.

Conveniently, the composition of the invention will be applied as a coating to the ‘wound-facing’ of the dressing but alternatively may be incorporated within the body of the dressing. However in other embodiments of wound dressing in accordance with the invention the polyanionic compound and the antimicrobial agent may be incorporated separately in the wound dressing (rather than in a single composition) and may be provided at different locations within the dressing. Several non-limiting embodiments of wound dressing in accordance with the invention are summarised below.
1. The wound dressing may be in the form of a fibrous dressing wherein the antimicrobial agent and the polyanionic compound are within the fibres.
2. The wound dressing may be in the form of a fibrous dressing wherein the antimicrobial agent is within the fibres and the polyanionic compound is applied to the surface of the fibres.
3. The wound dressing may be in the form of a fibrous dressing wherein the polyanionic compound is within the fibres and the antimicrobial agent is applied to the surface of the fibres. The antimicrobial agent may, for example, be a coating of metallic silver on the fibres.
4. The wound dressing may be in the form of a fibrous dressing and some or all of the fibres have an antimicrobial agent on the surface of the fibres and the polyanionic compound is applied to the surface of the fibres. The antimicrobial agent may, for example, be a coating of metallic silver on the fibres.
5. The wound dressing may be in the form of a foam dressing wherein the antimicrobial agent and the polyanionic compound are within the foam.
6. The wound dressing may be in the form of a foam dressing wherein the antimicrobial agent is on the surface of the foam and the polyanionic compound is within the foam.
7. The wound dressing may be in the form of a foam dressing wherein the antimicrobial agent is within the foam and the polyanionic compound is on the surface of the foam.
8. The wound dressing is in the form of a gel wherein the antimicrobial agent and the polyanionic compound are within the gel.

Typically dressings produced in accordance with the invention will comprise 0.01-20% by weight of each of the polyanionic compound and the antimicrobial agent, these percentages being based on the total weight of the dressing including the polyanionic compound and the antimicrobial agent. By way of more specific example, a dressing in the form of a gel may comprise 0.01-5% by weight (on the same basis) of a silver compound as the antimicrobial agent and 0.1-10% by weight of a polyphosphate. By way of a further specific example, a fibrous or foam dressing may comprise 0.1%-20% by weight of silver compound and 0.1-10% by weight of polyphosphate compound.

It will be appreciated that in the case of wound dressings in accordance with the invention which comprise fibres or fabrics the antimicrobial agent may be silver provided as a coating on the fibres or fabric.

Although the invention has been described so far with particular reference to the polyanionic compound and antimicrobial agent being, in effect, separate compounds it should be appreciated that the invention also extends to the combination of a polyanionic species and an antimicrobial species for the treatment of microbial biofilms and for the case where the polyanionic species and the antimicrobial species are part of the same chemical “entity”, e.g. a silver salt of a polyphosphate.

The invention will be illustrated by reference to Experimental Sections 1 and 2 below as well as the accompanying drawings, in which:

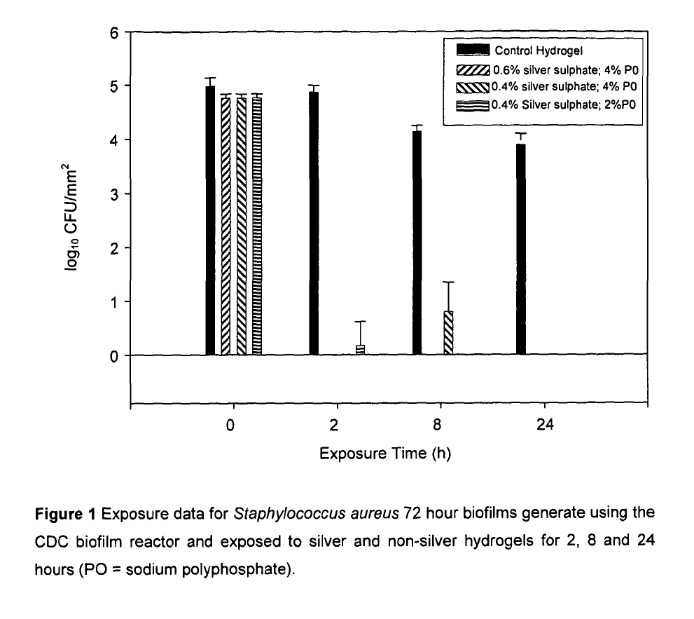

FIG. 1 is a graph of data obtained in accordance with Experimental Section 2 below and illustrating effectiveness of compositions in accordance with the invention for killing biofilms generated from Staphylococcus aureus;

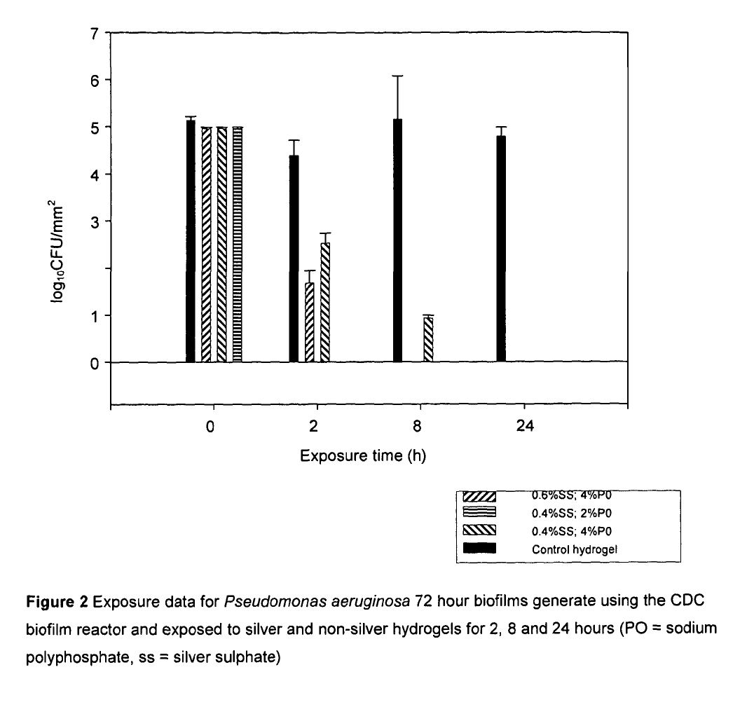

FIG. 2 is a graph of data obtained in accordance with Experimental Section 2 below and illustrating effectiveness of compositions in accordance with the invention for killing biofilms generated from Pseudomonas aeruginosa; and

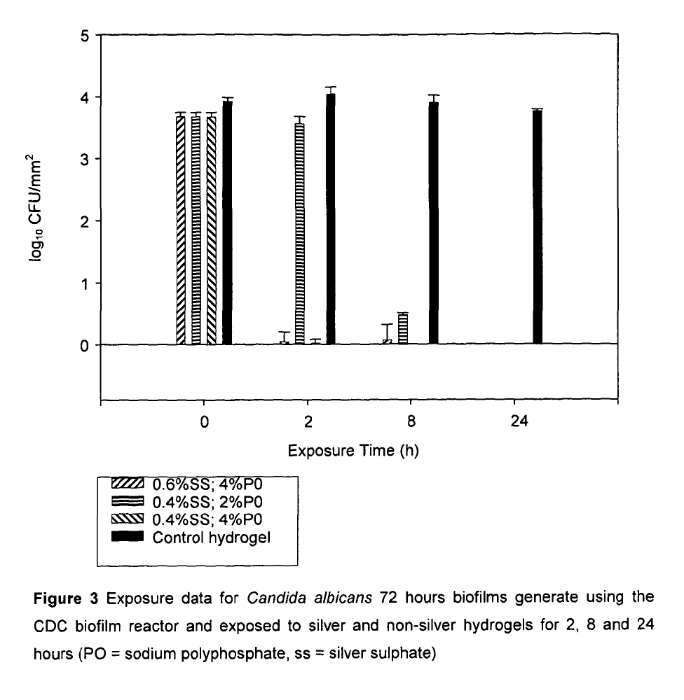

FIG. 3 is a graph of data obtained in accordance with Experimental Section 2 below and illustrating effectiveness of compositions in accordance with the invention for killing biofilms generated from Candida albicans.

### EXPERIMENTAL SECTION 1

### Methods

### Chemicals and Sterilisation of Growth Media

Unless otherwise stated all formulated bacteriological media were purchased from Oxoid (Basingstoke, Hampshire, UK). Bacteriological media and solutions were sterilised by autoclaving at 121° C. for 20 minutes (1 Kg/cm²). Glassware and disposable pipette tips were sterilised according to this protocol.

### Test Strains and Culture Conditions

The bacteria used in this study were Staphylococcus aureus ATCC6538, Candida albicans ATCC10231 and Pseudomonas aeruginosa ATCC9027. Pure cultures were maintained on nutrient agar at 37° C.

### Generation of Biofilms for Antimicrobial Challenge

The NUNC-TSP transferable solid phase screening system (NUNC, Roskilde, Denmark) was used to generate up to 96 reproducible biofilms in a microtitre plate. Briefly, a suspension of each bacteria was prepared in 0.9% saline solution and adjusted to a McFarland's standard no. 1.0. This bacterial suspension was then diluted 1:30 in sterile TSB and 150 μl aliquots aseptically transferred to a 96 well Calgary device. Plates were incubated for 24 and 72 hours and then submerged in 150 μl 0.9% saline solution to remove planktonic bacteria from the biofilm surface.

### Challenge of Bacterial Biofilms to Polyphosphate

A stock solution of polyphosphate (80 mg/ml) was prepared in distilled water and filter sterilised (0.22 μm). This was subsequently diluted 1:2 in triplicate across the wells in TSB to a final concentration of 0.04 mg/ml. Each well also consisted of positive controls (TSB only in absence of antimicrobial agent) and negative controls. NUNC solid phase lids were subsequently transferred to this challenge plate and incubated overnight (37° C.). Wells were visually inspected for turbidity signifying bacterial growth. The minimum inhibitory concentration was defined as the lowest concentration of polyphosphate to prevent bacterial growth. Aliquots of wells (10 μl) showing no visual signs of turbidity were transferred to fresh TSB stocks and incubated for a further 8 hours (37° C.). This allowed the determination of the minimum bactericidal concentration (MBC).

Subsequent to this, challenged NUNC peg lids were transferred to a 96 well recovery plate containing TSB media only. Plates were incubated overnight and visualised for bacterial growth. This allowed the determination of the minimum biofilm eradication concentration (MBEC). Any growth in the media corresponded to planktonic bacteria seeding the media from a viable biofilm. Absence of turbidity therefore signified bacterial eradication at this respective concentration of polyphosphate.

### Determination of Combinational Effects Between Ionic Silver and Polyphosphate

Biofilms were generated as described previously. Ionic silver (Silver carbonate, silver nitrate, silver sulphate; Sigma-Aldrich, Germany) was prepared in water (40 mg/ml) and filter sterilised (0.22 μm). Concentrations of polyphosphate were prepared horizontally across the NUNC 96 well plate, whilst ionic silver was prepared vertically. The range of concentrations used for each agent was equivalent to ×4 to 1/32 of the respective MIC. This permitted a diverse array of drug concentration combinations to be tested in a single investigation. The FIC index was used to determine whether each drug exhibited indifference or synergy when in combination. A synergistic effect was determined following an FIC of less than 0.5; an FIC of 0.5 to 2.0 was defined as indifference; whilst an FIC of greater than 2.0 was considered to be antagonistic.

### Results

TABLE 1
MIC, MBC, MBEC data following exposure of polyphosphate
to a 24 hour monospecies biofilm

| | MIC | MBC | MBEC |
|---|---|---|---|
| Bacteria | (mg/ml) | (mg/ml) | (mg/ml) |
| S. aureus | 1.25 | 10 | 40-80 |
| C. albicans | ng | ng | ng |
| P. aeruginosa | 5.0 | >80 | >80 |

Data are derived from NUNC biofilm exposures. Each exposure was performed in triplicate
ng signifies no growth

TABLE 2
Combinational effect data analyses between ionic silver (silver nitrate)
and polyphosphate following exposure to a 24 hour monospecies biofilm

| | MIC alone | MIC in | | MIC alone | MIC in | | |
|---|---|---|---|---|---|---|---|
| Bacteria | mg/ml (A) | combination (A) | FIC (A) | mg/ml (B) | combination (B) | FIC (B) | Outcome |
| S. aureus | 1.25 | 0.015 | 0.012 | 0.015 | 0.015 | 1 | Indifference |
| C. albicans | ng | ng | ng | ng | ng | ng | ng |
| P. aeruginosa | 5 | 0.625 | 0.125 | 10 | 0.625 | 0.0625 | Synergy |

Data are derived from NUNC biofilm exposures. Each exposure was performed in duplicate
ng signifies no growth

TABLE 3
Combinational effect analyses between ionic silver (silver nitrate)
and polyphosphate following exposure to a 72 hour monospecies biofilm

| | MIC alone | MIC in | | MIC alone | MIC in | | |
|---|---|---|---|---|---|---|---|
| Bacteria | mg/ml (A) | combination (A) | FIC (A) | mg/ml (B) | combination (B) | FIC (B) | Outcome |
| C. albicans | 5 | 0.03 | 0.006 | 0.07 | 0.03 | 0.428 | Synergy |
| P. aeruginosa | 80 | 40 | 0.5 | 0.6 | 0.07 | 0.1 | Indifference |

Data are derived from NUNC biofilm exposures. Each exposure was performed in duplicate

The MIC values for polyphosphate were 1.25 and 5 mg/ml when subjected to a 24 hour biofilm comprising Staphylococcus aureus and Pseudomonas aeruginosa, respectively, increasing to 20 and 80 mg/ml when exposed to 72 hour biofilms.
Synergy was observed between silver nitrate and polyphosphate against 24 hour P. aeruginosa biofilms (FIC index<0.5), but not when exposed to 72 hour P. aeruginosa biofilms. This result was, however, marginal with an FIC index of 0.6 (where synergy 0.5≧).
Synergy was also observed following exposure of polyphosphate and silver nitrate to 72 hour Candida albicans biofilm (FIC index<0.5).

### EXPERIMENTAL SECTION 2

This Experimental Section demonstrates the effectiveness of hydrogels containing various amounts of silver sulphate (ss) and sodium polyphosphate (PO) for killing biofilms generated with Staphylococcus aureus, Pseudomonas aeruginosa and Candida albicans. Hydrogel compositions tested comprised the following amounts of silver sulphate and sodium polyphosphate.

| | Silver Sulphate | Sodium Polyphosphate |
|---|---|---|
| Composition No. | (% by weight) | (% by weight) |
| 1 | 0.6% | 4% |
| 2 | 0.4% | 4% |
| 3 | 0.4% | 2% |
| Control | 0% | 0% |

### Methods

### Chemicals and Sterilisation of Growth Media.

Unless otherwise stated all formulated bacteriological media were purchased from Oxoid (Basingstoke, Hampshire, UK). Bacteriological media and solutions were sterilised by autoclaving at 121° C. for 20 minutes (1 Kg/cm²). Glassware and disposable pipette tips were sterilised according to this protocol.

### Test Strains and Culture Conditions.

The bacteria used in this study were Staphylococcus aureus ATCC6538, Candida albicans ATCC10231 and Pseudomonas aeruginosa ATCC9027. Pure cultures were maintained on nutrient agar at 37° C.

### Generation of Biofilms.

Bacterial biofilms were generated using the CDC biofilm reactor (BioSurface Technologies Corp, Montana, US). Briefly, polypropylene rods each housing polycarbonate coupons, were immersed into 400 ml TSB broth and conditioned overnight at room temperature. Following this, the biofilm reactor was inoculated with 1 ml respective test strain culture, previously adjusted to OD 1.5_{660 nm}. CDC biofilm reactors were maintained at steady state for 8 hours during which the waste pipe was clamped to prevent loss of media. The magnetic baffle bar was maintained at 125 RPM. Subsequent to this, the biofilm reactor was maintained at continuous flow at a rate of 10 cm³ TSB broth per hour for 72 hours.

### Exposure of Coupons to Silver Hydrogel.

Polycarbonate coupons were aseptically removed from the CDC biofilm reactor and transferred to sterile 12 well microtitre plates containing the respective hydrogel (3 cm³). Bacterial biofilms were exposed in duplicate for periods of 2, 8 and 24 hours at 37° C. Subsequent to this, coupons were transferred to 10 ml neutralisation buffer (0.1% Na₂S₂O₃, 1M CaCl₂ in dH₂O) and agitated in a Griffin shaker for 60 seconds, followed by 30 seconds pulse-vortex mixing. Suspensions were serially diluted 1 in 10 to a final dilution of 10⁻⁷ and plated in triplicate onto nutrient agar for incubation (0/N; 37° C.). All colony counts were recorded as log₁₀CFU/mm². Exposure data was expressed graphically using Sigma Plot 8.0 (Systat Software Inc., London, UK).

The results are shown in FIGS. 1-3 of the accompanying drawings and are discussed below.

It will be seen from FIGS. 1-3 that the control hydrogel compositions (containing no silver sulphate or sodium polyphosphate) did not provide any significant reduction in CFU/mm² (P>0.05).

FIGS. 1-3 do however clearly demonstrate that significant reductions in log CFU counts were obtained following 24 hours exposure of 72 hour old mono-species biofilms to all anti-biofilm hydrogels (i.e those containing both silver sulphate and sodium polyphosphate). Of particular note is that the anti-biofilm gel containing 0.4% Silver sulphate and 2% sodium polyphosphate gave a 5 log reduction in CFU/mm² (P<0.05) following 2 h exposure to P. aeruginosa; and the anti-biofilm gel containing 0.6% silver sulphate and 4% sodium polyphosphate gave a 5 log reduction in CFU/mm² (P<0.05) following 2 h exposure to S. aureus biofilms.
In general, all antimicrobial hydrogels containing silver and sodium polyphosphate exhibited significant anti-biofilm killing (based on log reduction of CFU/mm2 following 24 hours exposure) within 2 hours exposure.
Notable examples included silver gels with 0.4-0.6% silver sulphate (supplemented with 4% polyphosphate) inducing on average 5 log reductions in CFU/mm2 following only 2 hours exposure.

### REFERENCES

1. Costerton, J. W., et al. (1999) Science 284: 1318-1322.
2. Donlan, R. M. (2002) Biofilms: microbial life on surfaces. Emerg Infect Dis 8:881-90.
3. Gjodsbol, K., Christensen, J. J., Karlsmark, T., Jorgensen, B., Klein, B. M., Krogfelt, K. A. (2006) Multiple bacterial species reside in chronic wounds: a longitudinal study. Int Wound J 1:1-2.
4. Gottrup, F. (2004) A specialised wound-healing center concept: importance of a multidisciplinary department structure and surgical treatment facilities in the treatment of chronic wounds. Am J Surg 187:38 S-43S.
5. Van Wazer, J. R. and Callis, C. F. (1958) Metal complexing by phosphates. Chem Rev 58:1011-1046
6. Vaar, M. And Jaakkola, J. (1989) Sodium hexametaphosphate sensitizes P. aeruginosa, several other species of Pseudomonas and Escherichia coli to hydrophobic drugs Antimicrobial agents and chemotherapy 33:1741-1747

1. The combination of a first species which is a polyphosphate and a second species which is an antimicrobial agent for the treatment of microbial biofilms or for the topical treatment of wounds.

2.-4. (canceled)

5. The combination of claim 1 wherein the polyphosphate is an alkali metal polyphosphate.

6. The combination as claimed in claim 5 wherein the polyphosphate is sodium hexametaphosphate.

7.-9. (canceled)

10. The combination according to claim 1 wherein the antimicrobial agent is selected from metallic silver, silver compounds, iodine, PHMB (polyhexamethylene biguanide), acetic acid, chlorhexidine, aminoglycosides (e.g. amikacin, gentamicin, streptomycin and tobramycin), ansamycins, carbacephem, cephalosporins, glycopeptides (e.g. vancomycin), macrolides (eg clarithromycin), monobactams and sulfonamides).

11. The combination according to claim 10 wherein the antimicrobial agent is a silver compound.

12. The combination as claimed in claim 11 wherein the silver compound is selected from silver sulphate, silver carbonate, silver nitrate, silver chloride, silver oxide, silver citrate, silver hydrogen citrate, silver dihydrogen citrate, silver salts of EDTA (ethylenediaminetetraacetic acid) and silver sodium hydrogen zirconium phosphate.

13. The combination as claimed in claim 10 wherein the antimicrobial agent is nano-crystalline silver.

14. The combination as claimed in claim 10 wherein the antimicrobial agent is metallic silver and said combination comprises fibres and/or a fabric coated with said metallic silver.

15. A composition comprising the combination as claimed in claim 1.

16. A composition as claimed in claim 15 which is topically administrable.

17. A composition as claimed in claim 16 for the topical treatment of wounds.

18. A topically administrable wound treatment composition comprising a first species which is a polyphosphate and a second species which is an antimicrobial agent.

19. A composition as claimed in claim 18 which comprises 0.1-200 mg/ml of the polyanionic compound and 0.01 μg to 250 mg/ml of the antimicrobial agent.

20. A composition as claimed in claim 18 in the form of a liquid, powder, emulsion, cream, lotion, gel oil, ointment, gel, semi-solid formulation, or aerosol spray.

21. A composition as claimed in claim 18 for use in inhibiting microbial biofilm formation in or on a wound.

22. A composition as claimed in claim 18 for use in disrupting existing microbial biofilm formation in or on a wound.

23. A wound dressing comprising a substrate to which is applied a composition as claimed in claim 18.

24. A wound dressing comprising a substrate together with the combination as claimed in claim 1.

25. A wound dressing as claimed in claim 23 wherein the substrate is a layered dressing in which one or more layers of the dressing are formed at least in part or one or more layers of the dressing are formed, at lest in part, by one or more of; natural fibres, cellulose, cotton, Rayon, Nylon, acrylic, polyester, polyurethane foam, hydrogels, hydrocolloids, polyvinyl alcohol, starch, a starch film, a biodegradable material, and combinations thereof.

26. A wound dressing as claimed in claim 23 wherein the substrate comprises fibres and/or a fabric coated with metallic silver as the antimicrobial agent.

27. (canceled)

28. A method of inhibiting a wound infection, comprising administering to the wound a first species which is a polyphosphate and a second species which is an antimicrobial agent other than a polyanionic compound.

29. A method as claimed in 28 for inhibiting, reducing, removing biofilm on a wound.

30. A method as claimed in 28 effected using a composition comprising said polyphosphate and said antimicrobial agent.

31. The combination of a polyphosphate and an antimicrobial species for the treatment of microbial biofilms.
